(19) United States
(12) Patent Application Publication
(10) Pub. No.: US 2014/0248695 A1
(43) Pub. Date: Sep. 4, 2014

(54) Viral Vectors Purification System

(71) Applicant: MolMed, S.p.A., Milan (IT)

(72) Inventors: Chiara Bovolenta, Milan (IT); Anna Stornaiuolo, Milan (IT)

(73) Assignee: MolMed, S.p.A., Milan (IT)

(21) Appl. No.: 14/349,531

(22) PCT Filed: Oct. 5, 2012
(86) PCT No.: PCT/EP2012/069713

§ 371 Date: Apr. 3, 2014

(30) Foreign Application Priority Data
Oct. 5, 2011 (EP) EP 11183937.9

(51) Int. Cl. C12N 7/00; C07K 14/71

(52) U.S. Cl. CPC C12N 7/00; C07K 14/71
USPC 435/320.1; 530/350

(57) ABSTRACT

The present invention relates to a new method for purification of viral vectors particularly those belonging to the Retroviridae family, which is based on the expression in the packaging cell line that produced such vectors of an exogenous gene encoding a cell surface marker. The incorporation of the cell surface marker in the viral envelope of the vector allows purification with immunological methods.

### FIELD OF THE INVENTION

The present invention relates to a method for efficient purification of viral vectors (W) particularly those belonging to the Retroviridae family. More particularly the invention relates to the purification of VV by an immunological method based on the expression of an exogenous cell surface marker in the packaging cell line.

### BACKGROUND

Viral vectors are commonly used to deliver genetic material into target cells. Nowadays VV are used in gene therapy applications to vehicle therapeutic genes into patients. In clinical applications, it is necessary to develop high quality VV in order to meet requisites imposed by regulatory agencies. Particularly, it is necessary to develop safer producer cell lines, to be used in large-scale production processes in order to obtain large viral stocks. In the meantime, cost-efficient and scalable purification processes are essential for the production of clinical grade viral particles to be administered in humans.

Purification of VV preparations is mandatory to prevent toxicity, inflammation or immune response due to vector components, cellular and medium contaminants such as for example serum (Baekland et al., 2003; Tuschong et al., 2002). Ideally a purification process needs to assure maintenance of viral infectivity (stability), high recovery of viral particles, removal of contaminants such as DNA, proteins and inhibitors of transduction, possibility to concentrate viral supernatant and, of course, scalability of the process (Andreatis et al., 1999; Lyddiatt and O'Sullivan, 1998).

As of today, different procedures for the purification of retroviral vectors have been developed based on different technologies, particularly: centrifugation based methods, membrane separation processes, chromatographic or other methods based on precipitation with salts and polymers such as PEG. Currently proposed purification schemes result in low recovery (approximately 30%) (Rodrigues et al., 2007). All these methods have been developed originally for protein production and, further, they have been adapted to the purification of VV. Due to the peculiarity and complexity of viral particles, it is necessary to improve purification methods in order to obtain high productivity and high throughput while maintaining the biological activity of the final product, particularly in terms of infectivity.

A further and more recent example is reported in Merten et al., 2011 that disclose a process for the production of lentiviral vectors in large scale and under GMP conditions, to be used in the context of a pilot gene therapy clinical trial for the treatment of Wiskott-Aldrich syndrome. The disclosed process includes both production and downstream processes for the purification of lentiviral particles. Particularly, the purification is based on a multistep scheme combining several chromatographic and membrane based process steps including anion exchange and size exclusion chromatography. Notwithstanding the very good results in respect to the production rate and the absence of DNA and protein contaminants in the final preparations, the final yield of the purification process is in the range of previously disclosed methods (below 30%) and the infectivity of the viral vectors in the final sample is reduced.

Both viral and cellular proteins are incorporated into the viral envelope during viral maturation and release from host cells and, in particular during the so called budding process (Arthur et al., 1992). It has been shown, for example, that numerous endogenous host cell proteins are incorporated into the HIV-1 envelope including human lymphocyte antigens, (HLA) classes I and II, CD44, complement control proteins and others whereas others, such as CXCR4, CCR5 and CCR3, are excluded. On the basis of this observation, it was suggested that cell type specific antigens may serve as marker of the cellular origin of HIV-1 replication (Roberts et al., 1999). Furthermore, it was developed an immunomagnetic viral capture assay that was able to distinguish between lymphocytes derived and macrophages derived propagated HIV viruses (Lawn et al., 2000). Particularly, Lawn et al. showed that it was possible to isolate T-cell-derived HIV viruses using an antibody able to bind CD26, and to discriminate it from macrophage-derived HIV-1 viruses that, in turn, can be captured using anti CD36 antibodies. Both CD26 and CD36 are endogenous host cell proteins that are over-expressed during HIV-1 infection in T-cells and macrophages, respectively. Both proteins are also incorporated in the viral envelope thus allowing selective isolation of the virus. Lawn et al. tested a panel of antibodies able to bind host cell specific antigens before identifying the successful ones. Interestingly, several antibodies able to bind antigens endogenously expressed at high level by macrophages (CD32, CD64, CD88 and CD89) are instead not able to capture the virus, thus showing that over-expression of a certain marker on the surface of the host cell is a necessary but not sufficient condition for capturing the virus. Lawn et al. do not show that exogenous proteins expressed by the host can be successfully incorporated into the virus envelope, and subsequently used for the purification of functionally active viral particles.

It has been shown that certain modified cell surface markers can be used for the purification of transduced cells. Particularly, WO/9506723 discloses a process of marking eukaryotic (mammalian) cells by expressing in these cells the nucleic acid encoding a cell surface receptor, that is further presented at the cell surface. This cell selection process is characterized by the use of a nucleic acid in which the region encoding the intracellular domain of the receptor is completely or partly deleted, or modified so that the receptor presented at the surface cannot effect any signal transduction after binding to its binding partner. The cell surface receptor employed in the disclosed process is the Low Affinity Nerve Growth Factor Receptor (LNGFR), in a truncated form in which the intracellular domain has been deleted. The resulting truncated cell surface receptor is called ΔLNGFR. The presence of the ΔLNGFR protein allows the in vitro immunoselection of the genetically modified cells through the use of monoclonal antibodies and magnetic beads.

ΔLNFGR is a truncated cell surface marker that is currently employed in gene therapy for the selection of transduced cells. For example, it is employed in the HSV-TK gene therapy approach, which enables safe haploidentical haematopoietic stem cell transplantation (HSCT) for the treatment of haematological malignancies. The TK therapy employs a retroviral vector which carries both the suicide gene HSV-TK and the marker gene ΔLNGFR (Verzeletti et al. 1998).

So far, the ΔLNGF receptor has not been employed for the purification of VV.

Due to the necessity of producing purified VV for clinical applications, several attempts have been made to obtain efficient purification processes that allow good recovery of VV as well as generation of sufficiently safe vectors that still have good quality in terms of stability. The methods which are currently employed, allow to obtain low recovery and have some limits in any case, since they derive from downstream processes developed for the production of recombinant proteins and adapted to VV purification. Therefore, there is a need of efficient, fast and scalable purification methods for VV to be used for large scale production of vectors for gene therapy, that allow to obtain good recovery and safe viral particles which maintain high infectivity.

### SUMMARY OF THE INVENTION

The present invention relates to the field of purification of VV, particularly those belonging to the Retroviridae family. Downstream processes currently employed for the purification of VV are based on methods usually applied for recombinant proteins. VV are peculiar particles that are employed in basic research and gene therapy clinical trials and that, in the latter case, need clinical grade production. Purification is therefore mandatory but, due to the peculiarity of VV, there are several necessities that need to be satisfied. The purification process needs to be efficient and fast since VV are sensitive to environmental conditions, and needs to be scalable since large batches are required in clinical applications.

The present invention provides a new strategy for the purification of viral particles, that is based on the exploitation of the property of such particles to incorporate host cell proteins embedded in the cellular plasma membrane into their external envelope. The purification method consists in the expression of an exogenous gene encoding a cell surface marker in the packaging cell line for the production of the VV. Such marker is exposed on the cellular membrane of the packaging cells. In the course of the production of the viral particles, during the maturation phase, the cell surface marker is incorporated into the viral envelope through the budding process. When incorporated into the viral envelope, the maker is, in fact, a viral surface marker, but we shall continue to refer to said marker as “cell surface marker”. The viral particles can be then incubated with an antibody able to recognize such marker and purified through immunological methods. All cell surface proteins that are exogenous to the packaging cells, particularly to packaging epithelial cells, can be employed as cell surface markers in the present invention. Cell surface markers that can be employed are for example CD26, CD36, CD44, CD3, CD25 and the Low Nerve Growth Factor Receptor in which the intracellular domain has been deleted (ΔLNGFR). In a preferred embodiment the cell surface marker that is used for the purification process is ΔLNGFR.

The developed purification method is extremely versatile since it is applicable to any VV that incorporates the proteins of the host cell membrane during the maturation through the budding process, such as retroviruses, lentiviruses, alpha viruses [e.g. Semliki Forest virus (SFV), Sindibis virus (SIN)], rhabdoviruses [e.g. vesicular stomatitis virus (VSV)], and orthomyxoviruses [e.g. influenza A virus]. Moreover, the purification method is linked to the production method because it requires expression of the marker in the packaging cell line and, therefore, allows an integrated approach in which production and downstream processes are built on the same starting material (the packaging cell line). In this context, it is possible to produce stable packaging cell lines containing all elements necessary for the production of VV as well as a further exogenous gene encoding the cell surface marker necessary for the purification. This aspect is particularly useful for both scalability and efficiency.

The method is based on the use of a ligand able to bind the cell surface marker, in order to separate VV from supernatant. Preferably the ligand is an antibody and the separation of viral particles is obtained by immunological methods. More preferably the method employs immunomagnetic selection. The method of the invention can be easily scaled-up and automated since several instruments exist for the performance of immunomagnetic selection.

Moreover the proposed method is handily, very fast and extremely efficient: the purification efficiency is higher than that obtained with the currently used methods, for example chromatographic methods such as those employing DEAE and SEC columns.

In addition, it has been found that the purification method of the invention allows high recovery, since it has been shown that the titer yield of vectors purified through this method is at least 85% or even higher (120%) in most experiments in small scale. Moreover, a considerable increase of infectivity of lentiviral vectors purified with the method of the invention has been obtained (43% and 60% for transient and stable production, respectively), in small scale experiments. Such titer yield and infectivity increase are achieved with the single main step of the purification method of the invention i.e. the separation of the complex viral vector-ligand, without taking into consideration other steps that could affect the final results.

Large scale experiments have also been performed and very good results have been obtained. In fact, the recovery in terms of virus titer is 60%, with the single step of separation of the complex viral vector-ligand. Before starting the separation phase, the viral supernatant is enriched in its viral titer through preliminary filtration and centrifugation steps, that roughly eliminate contaminants and transduction inhibitors, and also through the incubation with the ligand of the receptor. The final titer yield after the complete multi-step purification process (harvest viral supernatant vs final purified product) results to be more than 100%. These are very good results considering that the titer yield of the complete purification processes disclosed in the literature is around 30% (Rodrigues et al. 2007) or even below in the case of large scale preparations (Merten et al. 2011). Moreover, VV purified according to the method of the invention on a large scale result to have a three times increase of the infectivity, an even higher increase than that obtained with the same method on a small scale. This is a very important and unexpected advantage since the literature describes a decrease of infectivity further to the purification with conventional methods (Merten et al. 2011).

Statements of the Invention

According to a first aspect of the invention there is provided a method for the purification of a viral vector comprising:
i. introducing an exogenous gene encoding a cell surface marker and a gene of interest (GOI) in a packaging cell line
ii. culturing the so obtained producer cell line
iii. collecting the supernatant containing viral vector particles bearing the cell surface marker on their external envelope
iv. incubating said supernatant with a ligand able to bind to the cell surface marker
v. separating complex ligand-viral vector
vi. obtaining purified viral vector particles

In another aspect of the invention the supernatant containing viral vector particles bearing the cell surface marker on their external envelope is filtered, optionally concentrated and then incubated with a ligand able to bind to the cell surface marker.

Preferably the viral vector is a retroviral vector, a lentiviral vector, an alpha viral vector [e.g. a vector obtained from Semliki Forest virus (SFV), Sindibis virus (SIN)], a rhabdoviral vector [e.g. a vector derived from vesicular stomatitis virus (VSV)], and an orthomyxoviral vector [e.g. a vector derived from influenza A virus]. More preferably the viral vector is a lentiviral vector or a retroviral vector.

In one embodiment the cell surface marker is any cell surface marker that is exogenous to the packaging cell line, which is, preferably, an epithelial packaging cell line. Preferably the cell surface marker is selected from CD26, CD36, CD44, CD3, CD25 and ΔLNGFR.

More preferably the cell surface marker is ΔLNGFR.

In one aspect of the invention the expression of the cell surface marker is transient.

In another aspect of the invention the expression of the cell surface marker is stable.

In one embodiment the GOI and the cell surface marker are expressed in the same transfer vector.

In another embodiment the GOI and the cell surface marker are expressed in separate vectors.

Preferably the ligand is a chemical or a biological entity selected from an agonist, an antagonist, a peptide, a peptidomimetic, an antibody, an antibody fragment, an affibody.

In a further aspect of the invention the ligand is linked to a moiety that can be separated from the supernatant.

Preferably the ligand is an antibody.

More preferably the antibody is conjugated to magnetic beads and separation is obtained by applying a magnetic field to a solution containing the complex antibody-viral vector.

Preferably the viral vectors are obtained by removing the magnetic field. More preferably the separation of the complex antibody-viral vector is performed on a column and the viral vectors are obtained by removing the magnetic field and further eluting them from the column.

In one embodiment the viral vectors are separated from the antibody by cleaving the cell surface marker-antibody bond.

In another aspect of the present invention there is provided an exogenous cell surface marker expressed in a packaging cell line for use in the purification of viral vectors produced by said packaging cell line.

Preferably the viral vector is a retroviral vector, a lentiviral vector, an alpha viral vector [e.g. a vector obtained from Semliki Forest virus (SFV), Sindibis virus (SIN)], a rhabdoviral vector [e.g. a vector derived from vesicular stomatitis virus (VSV)], and an orthomyxoviral vector [e.g. a vector derived from influenza A virus]. More preferably the viral vector is a lentiviral vector or a retroviral vector.

The cell surface marker is exogenous to the packaging cell line, preferably exogenous to epithelial packaging cells. Preferably the cell surface marker is selected from CD26, CD36, CD44, CD3, CD25 and ΔLNGFR.

More preferably the cell surface marker is ΔLNGFR.

In one aspect of the invention the expression of the cell surface marker is transient.

In another aspect of the invention the expression of the cell surface marker is stable.

### DETAILED DESCRIPTION OF THE INVENTION

A detailed description of preferred features and embodiments of the invention will be described by way of non-limiting example.

The invention can be put into practice by a person of ordinary skill in the art who will employ, unless otherwise indicated, conventional techniques of chemistry, molecular biology, microbiology, recombinant DNA and immunology. All such techniques are disclosed and explained in published literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); Current Protocols in Immunology, ch. 12, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, In Press; and, D. M. J. Lilley and J. E. Dahlberg, 1992, Methods of Enzymology: DNA Structure Part A: Synthesis and Physical.

Analysis of DNA Methods in Enzymology, Academic Press. All these publications are incorporated by reference.

Purification Method

The present invention provides a new purification method for VV. Preferably the invention relates to a method for the purification of VV including gamma retroviruses (prototype: Moloney murine leukemia virus, Mo-MLV), lentiviruses (prototype: HIV), alpha viruses [e.g. Semliki Forest virus (SFV), Sindibis virus (SIN)], rhabdoviruses [e.g. vesicular stomatitis virus (VSV)], and orthomyxoviruses [influenza A virus]. The proposed purification method is based on one of the phases of maturation of viruses. Viral particles are secreted from host cells through the budding process by which process viral capsid is wrapped with the plasma membrane derived from virus producer cells. In doing so, viruses incorporate in their external envelope several host cell proteins which are normally embedded in the cellular plasma membrane.

The VV produced by either a transient or stable packaging system are released from the packaging cells in an identical manner. The method of the present invention is based on the hypothesis that VV can be specifically purified by using an antibody directed against an exogenous host plasma membrane protein.

According to a first aspect of the invention there is provided a method for purification of a VV, that is based on the expression of an exogenous gene encoding a cell surface marker in a packaging cell line. The cell surface marker is a protein exogenous to the packaging cells that is expressed on the cellular membrane. Once expressed, such marker is mounted on the packaging cell membrane and, therefore, during the maturation of the VV, is mounted on the external envelope of the VV produced by said packaging cells. According to the method of the invention, the supernatant containing viral particles embedding a cell surface marker on their envelope is collected and incubated with a ligand able to recognize such marker. Optionally, particularly for the purification of VV on a large scale where large volumes have to be handled and purified, the supernatant is filtered and concentrated, and then resuspended and incubated with the ligand. All these preliminary steps allow a rough removal of contaminants and transduction inhibitors and, therefore, contribute to the enrichment in viral titer observed in the intermediate preparations and, consequently, to the final titer of the purified viral particles. Further to these preliminary steps, the complex ligand-VV is separated from the medium and VV are then obtained. The separation phase is the main step of the purification method according to the invention. The VV can be separated from the ligand upon cleavage of the bond between the ligand and the cell surface marker.

In a preferred embodiment the ligand is an antibody.

The method of the present invention can be summarized in five main phases:
1) Expression of a cell surface marker in a packaging cell line for VV
2) Production of viral particles
3) Incubation with a ligand able to recognize the cell surface marker
4) Separation of the complex ligand-receptor
5) Recovery of purified viral particles

Expression of the Marker and Production of Viral Particles

The purification method of the present invention is based on the expression of an exogenous cell surface marker in the packaging cell line for the VV. In a preferred embodiment the cell surface marker is exogenous to epithelial packaging cells. Preferably the cell surface marker is selected from CD26, CD36, CD44, CD3, CD25 and the truncated form of Low Nerve Growth Factor Receptor missing the intracellular domain (ΔLNGFR). In a preferred embodiment the cell surface marker is ΔLNGFR. The expression of the cell surface marker can be obtained in several ways. In one aspect of the invention, the cell surface marker can be transiently expressed in the packaging cell line. In one embodiment the cell surface marker and the therapeutic gene are both expressed in the same transfer vector. In another embodiment the cell surface marker and the therapeutic gene are expressed in separate vectors. In another aspect of the invention the expression of the cell surface marker is stable. The invention therefore provides a packaging cell line containing all structural elements necessary for the production of VV such as viral gag/pol, rev, optionally tat and the envelope protein of interest, together with the cell surface marker. In one embodiment, all these genes are stably integrated into the stable packaging cell line. In another embodiment the elements necessary for the production of VV are transiently expressed. The packaging cell line can be used to produce VV further to the introduction of the transfer vector containing the GOI. This packaging cell line represents an integrated technical solution that contains all elements necessary for the production of the VV and allows a rapid, safe and efficient purification method.

Further to the introduction of the transfer vectors, the production is obtained by culturing the packaging cell line containing the stably integrated or transiently expressed cell surface marker. The viral particles incorporate the cell surface marker into their envelope during the budding process and they are released in the supernatant. Purified viral particles will be obtained by exploiting the presence of the exogenous cell surface marker as described above.

In another embodiment there is provided a producer cell line containing all structural elements necessary for the production of VV such as viral gag/pol, rev, optionally tat, the envelope protein of interest and the GOI, together with the cell surface marker. Following to the culturing of the producer cells, viral particles containing the cell surface marker are released in the supernatant and they are purified exploiting the presence of the exogenous cell surface marker as described above.

Incubation with Ligand and Separation of the Complex Ligand-Cell Surface Marker

Viral particles containing a cell surface marker incorporated into their envelope can be isolated. According to the purification method of the present invention, the supernatant containing VV is incubated with the ligand able to recognize the cell surface marker. In another embodiment the supernatant containing the VV is first filtered and concentrated and then is incubated with the ligand. The ligand is linked to another structure that allows separation from the supernatant and, consequently, isolation of the ligand-VV complex. The ligands that can be used in the present invention are chemical or biological entities including but not limited to agonists, antagonists, peptides, peptidomimetics, antibodies, antibody fragments, affibodies.

Preferably the ligand is an antibody and the method of the present invention comprises immunoselection for the separation of the complex antibody-VV. In this case, VV containing the cell surface marker on their envelope may be selected on the basis of their reactivity with the anti-cell surface marker antibodies.

More preferably, the method of the present invention comprises immunomagnetic selection. Immunomagnetic selection refers to the coupling of antibodies to paramagnetic microspheres (beads) enabling a separation of the antigenic structures by means of a magnet. For example, supernatant containing VV incorporating the cell surface marker into their envelope may be incubated with a primary IgG anti-cell surface receptor antibody. The retroviral supernatant may be then incubated with immunomagnetic beads coated with anti-IgG secondary Ab, and applied to a magnet in order to separate the retroviral vectors carrying the marker. After separation from retroviral supernatant, the isolated vectors may be recovered by removing the magnetic field. Alternatively, the anti-cell surface receptor antibodies can be directly conjugated to magnetic beads. In this case, immediately after the single incubation phase the magnetic field is applied to the solution.

Paramagnetic microsphere to be employed in the present invention are known in the art, they are polymer particle having small size ranging from 50 nm such as the commercially available MACS® microbeads from Miltenyi Biotec, to bigger particles of 0.5-500 μm such as the commercially available Dynabeads®, from Invitrogen. Paramagnetic microsphere can be directly or indirectly conjugated to the specific antibody of interest able to bind the cell surface marker incorporated into the VV envelope. Method of conjugation of antibody to paramagnetic beads are known in the art and include, for example, cross linking, formation of covalent bonds on functional groups, biotin-avidin system and others. Separation of the VV from viral supernatant will be obtained by applying a magnetic field to a solution containing the complex consisting of the antibody conjugated paramagnetic beads and linked to the cell surface marker.

In a preferred aspect of the invention immunomagnetic selection is performed on a column. Particularly, the supernatant containing VV may be directly incubated with an antibody able to recognize the cell surface marker, such antibody being conjugated to paramagnetic beads. In another embodiment the supernatant containing the VV is first filtered and optionally concentrated and then is incubated as previously described. Following incubation, the supernatant or the filtered and optionally concentrated solution is applied on a column placed in a magnetic separator for the removal of impurities and separation of the viral particles that remain in the column thanks to the magnetic field.

Recovery of Purified Particles

According to the method of the invention, the last phase of the process is the recovery of purified viral particles. Such recovery is obtained removing the viral particles from the magnetic field. If the purification is performed on a column the recovery is obtained by removing the magnetic field. Viral vectors can then be separated from the ligand by cleaving the bond between the ligand and the cell surface marker. Methods for cleaving said bond are known in the art and include the use of displacement ligands or of appropriate solutions containing enzymes. Depending on the nature of the ligand and of the receptor and their bond the appropriate method is employed.

Efficiency, Scale-Up and Automation

The method of the invention is extremely efficient and is simple and fast. Currently proposed purification methods allow a recovery around 30%. Remarkably the method of the invention allows to obtain titer yield of at least 85% or even higher (120%) in most experiments in small scale. The titer yield, in this case, has been calculated referring to the main step of the method of the invention: the separation of the complex viral vector-ligand. The above-mentioned titer yield results from the ratio between the titer of the unpurified particles incubated with the ligand of the exogenous receptor and the titer of the purified particles. The unpurified particles in experiments on a small scale are obtained from the VV supernatant through preliminary filtration step, and are then incubated with the ligand of the exogenous receptor. The results show that the recovery in terms of viral titer with the method of the invention is very high.

Viral particles are extremely labile and sensitive to environmental conditions. In particular, the presence of the cell surface maker on the envelope of VV could, in principle, affect the composition of such envelope as well as its structure and the availability of viral envelope proteins, affecting in turn the tropism of the vector and, consequently, causing problems to viral titer and infectivity. With the method of the invention, on the contrary, the titer is unaffected or even increased and, remarkably, the infectivity of lentiviruses purified with such method is increased (43% and 60% for transient and stable production, respectively) in small scale. These results are surprising because of the presence of structural elements that could, possibly, negatively affect the tropism and the infectivity of the vectors.

A further advantage of the invention is that the method can be simply scaled-up and automated. Considering the case in which VV are purified by using immunomagnetic selection, it is possible to employ machines (e.g. CliniMacs® Plus Instrument, from Miltenyi Biotec) able to perform automated selection. Such machines must be able to perform liquid exchange on a solid support such as a column and immunomagnetic selection through the generation of a magnetic field. Automation helps the production of large VV stocks since it allows the purification of large quantity of viral supernatant. Purification of VV on a large scale with the method of the invention allows to obtain at least 60% titer yield in the main specific separation phase (titer of the unpurified particles incubated with ligand of the receptor vs titer of the purified particles). The unpurified particles, in experiments on a large scale, are obtained from the VV supernatant through preliminary purification steps: filtrations, optionally concentration, and are then incubated with the ligand of the exogenous receptor. Each of these steps causes a rough removal of contaminants and transduction inhibitors, with a progressive enrichment in viral titer of the sample that undergoes the separation. The titer yield of the entire process on a large scale (titer of harvest viral supernatant vs titer of purified particles) results to be more than 100%. These are a very important results considering that the titer yield of an entire purification process of VV is reported to be around 30% (Rodrigues et al. 2007) or even below in the case of large scale preparations (Merten et al. 2011). The purification on a large scale with the method of the present invention allows an increase of the infectivity of VV that result to be about 3 times more infective than the unpurified particles after the separation. In addition, in order to have an idea of the quality of the preparation obtained with the purification method of the present invention, it is possible to count the number of the lentiviral infectious particles (Transfecting Units derivable from the viral titer) vs total physical particle (obtainable from the conventional equation that 1 ng of p24Gag corresponds to 10⁷ physical particles, as reported in Salmon and Trono, 2006). It is very interesting to note that, with the method of the invention, as shown for example in Experiment 2 of Table 4, starting from a supernatant containing 1 infectious particle out of 5,318 total physical particles (a very poor starting material), it was possible to obtain a purified preparation containing 1 infectious particle out of 250 total physical particles, with a very good enrichment in terms of quality and functionality of the LV preparation.

Further preferred features and embodiments of the present invention will now be described by way of non-limiting example and with reference to the accompanying drawings in which:

### DESCRIPTION OF THE FIGURES

FIG. 1. Schematic representation of the strategy of the anti-ΔLNGFR-Ab-based purification process. “Constructs” indicates the plasmids or vectors required to produce either transiently or stably the VV to be purified (step 1). The cassette of the selection marker ΔLNGFR can be incorporated into the transfer vector construct or in a different plasmid expressed either transiently or stably from the packaging cells. The VV are purified by the anti-ΔLNGFR Ab coupled to magnetic beads which are then retrained in a magnetic column (step 2) and finally eluted (step 3). The purified VV can be either used with the attached beads (step 3.1) or after removal of the attached beads (step 3.2).

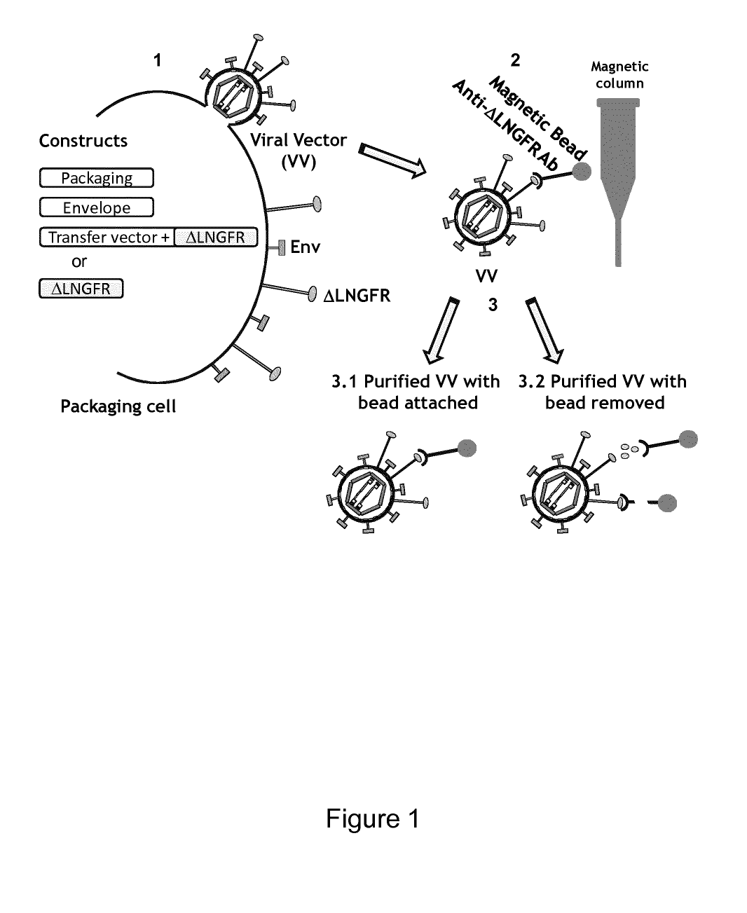

FIG. 2. Schematic representation of the experimental procedure of the invention. The procedure is divided in three simple steps: 1) the magnetic labelling of the VV consisting in the incubation of supernatants with the anti-ΔLNGFR Ab conjugated microbead suspension for 30 minutes at room temperature; 2) the magnetic separation of the VV consisting in the application of the sample into the magnetic column placed into the magnetic separator; all sample components (i.e contaminants, proteins and excess Abs) go in the flow-through and further removed by several washes; 3) the elution of the VV consisting in the removal of the column from the magnetic separator and collection of the purified VV. The purified VV can be either used with the attached beads (step 3.1) or after removal of the attached beads (step 3.2).

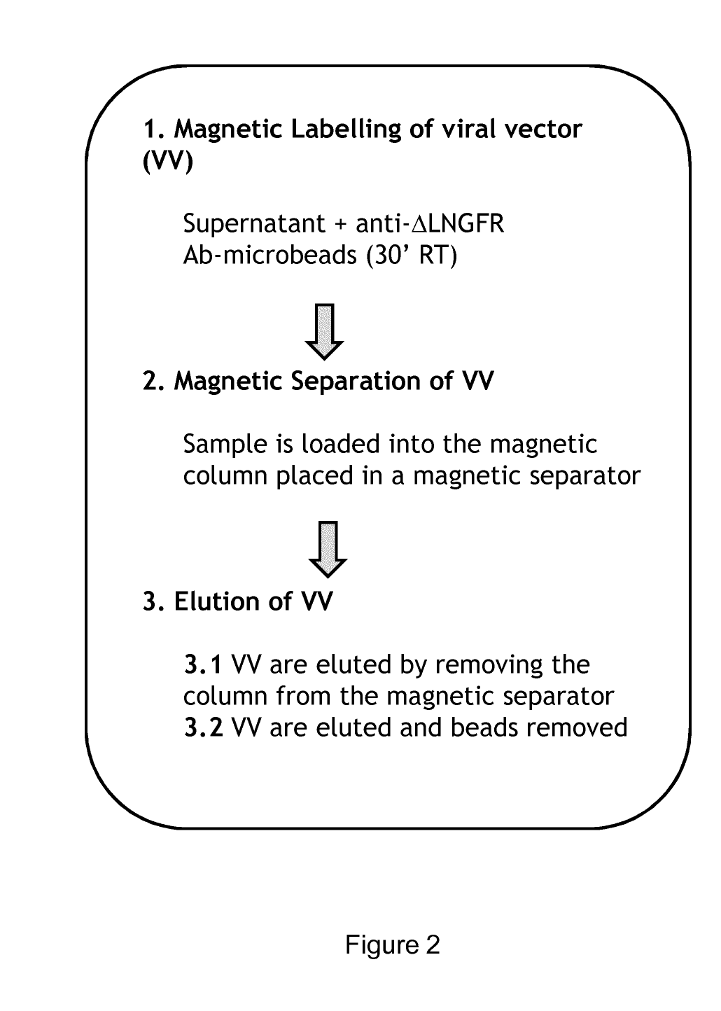

FIG. 3. Graph summarizing the experimental data in small scale. The yield of the VV titer has been calculated as percentage of the titer of the purified VV vs that of the magnetically labeled VV before loading the sample into the column. The values are the means of 5 experiments for the lentiviral vectors, produced stably by the RD2-MolPack-Chim3.14 packaging clone, which carry the RD114-TR envelope (Stable LV, RD114-TR); 6 experiments for the lentiviral vectors, produced by transient transfection of HEK-293T, which carry the VSV-G envelope (Trans. Transf. LV, VSV-G); 5 experiments for the retroviral vectors, produced from the AM12-SFCMM-TK clone 48, which carry an Ampho envelope (Stable RV, Ampho).

### EXAMPLES

### Example I

### Production of VV

Stable Production of MLV Retroviral Vectors (RV)

Murine NIH-3T3-derived, e4070-pseudotyped AM12-SFCMM-TK clone 48 packaging cells were grown in DMEM (Dulbecco's Modified Eagle Medium) (BioWhittaker™, Cambrex Bio Science Walkersville, Inc. Walkersville, Md.) or X-VIVO 15 supplemented with 10% FBS (BioWhittaker™) and 2 mM glutamine at 37° C. in 5% CO₂ atmosphere. The AM12-SFCMM-TK clone 48 was obtained after transduction of the construct SFCMM-3 Mut2, which encodes a modified form of the HSV-TK gene characterized by a single silent mutation at nucleotide 330 of the ORF (WO 2005/123912). Transduced cells were immune selected by using the anti-ΔLNGFR mAb of the Am12-SFCMM-3 Mut 2 cells and then cloned by limiting dilution (0.3 cell/well). AM12-SFCMM-TK clone 48 contains two copies of SFCMM-3 Mut2 vector. The GMP-grade retroviral vector supernatant lots were produced either in roller bottles or in a packed-bed 32-liter bioreactor in X-VIVO 15 medium in the presence of 1% glutamine, 10% PBS and isoleucine/tryptophan/Na citrate.

Stable Production of Lentiviral Vectors (LV)

Human HEK-293T and its derivative RD2-MolPack-Chim3 packaging cells were propagated in Dulbecco's Modified Eagle Medium (DMEM) supplemented with 10% FCS and PSG. RD2-MolPack-Chim3.14 and Chim3.25 clones stably produce second generation LV for anti-HIV gene therapy approach. The clones were obtained by sequential integration of the packaging constructs and the transfer vector using integrating vectors. Briefly, HEK-293T cells were transiently transfected with a plasmid encoding the adeno-associated virus (AAV) Rep-78 protein and then infected with an hybrid baculovirus-AAV vector, in which the baculoviral backbone contains an integration cassette expressing the HIV-1 structural gag, pol, the regulatory rev and the hygro-resistance genes flanked by the AAV inverted terminal repeats (ITR) sequences (International Patent Application N° WO 2012/028680). This system, which allows the Rep78-mediated integration of the ITR-flanked cassette into HEK-293T genome, generated the first intermediate clone named PK-7. From PK-7 clone, the RD2-MolPack-Chim3.14 and Chim3.25 packaging clones (International Patent Application N° WO 2012/028681) were obtained through the sequential integration of the SIN-LV expressing the HIV-1 regulatory tat and the chimeric RD114-TR envelope gene and the Tat-dependent LV vector expressing the anti-HIV Vif dominant negative transgene Chim3. Clones were obtained by seeding the cells at limiting dilution in 96-well plate (0.1 to 0.3 cell/well). For each cell type cloning experiment, at least 5 to 10 individual clones or more were selected by visual inspection under optical microscope and gradually expanded. LV derived from RD2-MolPack-Chim3 were obtained by small scale culture in either T25 or T75 flasks and by large scale in 1162 flasks

Transient Production of LV

Pseudotyped LV produced from HEK-293T cells were obtained by transient co-transfection of the following plasmids: the packaging constructs CMV-GPRT, the VSV-G construct, and the 2^{nd}-gen. PΔN-Chim3 transfer vector (International Patent Application N° WO 2012/028681). The ratio of packaging:envelope:transfer vectors corresponded to 6.5:3.5:10 μg DNA. Transient transfections were performed with either the standard Ca2⁺-PO4 method or the Fugene™6 system following the manufacturer's instruction (Roche Diagnostics Corporation, Indianapolis, Ind.) obtaining similar results. Supernatants were harvested 48 hours after transfection and filtered through a 0.45-μm filter.

### Example II

### Purification of VV by the Anti-LNGFR Abs on a Small Scale

Small scale purification of VV was carried out as follows. Supernatants containing VV were diluted with 1:5 (vol/vol) with PBS containing 0.5% BSA and then filtered with 0.45 μm filters. From one to five ml of diluted supernatants were incubated with anti-LNGFR Ab conjugated microbeads suspension (CD271 Microbeads Miltenyi Biotec, GmbH, Germany cat. #130-091-330) at the 1:40 ratio (vol/vol). The samples were then incubated at room temperature (RT) for 30 minutes on a rotating wheel. The magnetically labelled samples were loaded on the column placed into the magnetic separator, (Miltenyi, MS Columns cat. #130-042-201). After the flow-through was collected for analysis and three washes were performed with 0.5 ml of washing buffer (PBS containing 2% FCS and 0.5% BSA), the column was removed from the magnetic separator and the purified VV were collected.

### Example III

### Titer Calculation

VV titer was calculated on SupT1 cells by transducing them by one cycle of spinoculation at 1,240×g for 1 hour in the presence of polybrene 8 μg/ml (Sigma-Aldrich, St Louis, Mo.). Transduction efficiency was monitored by flow cytometry analysis (FACS Calibur BD Bioscience, San Jose, Calif.) of ΔLNFGR expression, as described in Porcellini et al., 2009 & 2010, using the FlowJo software (Tree Star, Inc., Ashland, Oreg.). Only transduction values ranging from 5 to 20% positive cells were used to calculate the titer according to the formula: TU=[number of cells×(% positive cells/100)]/vol sup (in ml).

### Example IV

### Analysis of Potency of Purified Versus Unpurified VV in Small Scale Preparation

Several experiments were performed, as summarized on Table 1, and Table 2, using three types of VV produced by different modalities and pseudotyped with distinct envelopes. The 2^{nd} generation LV expressing the Chim3 transgene were produced from either the stable packaging cell line RD2-MolPack-Chim3.14 or by transient transfection of HEK-293T cells as reported in Example I. In the first condition, the LV were pseudotyped with the chimeric RD114-TR envelope, made of the extracellular and trans-membrane domains of the feline endogenous retrovirus RD114 envelope and the cytoplasmic tail (TR) of the A-MLVenv 4070A (Sandrin et al., 2002), whereas in the second condition with the vesicular stomatitis virus glycoprotein G (VSV-G) envelope. The γRV were produced from the AM12-SFCMM-TK clone 48 and carried the MLV e4070 envelope.

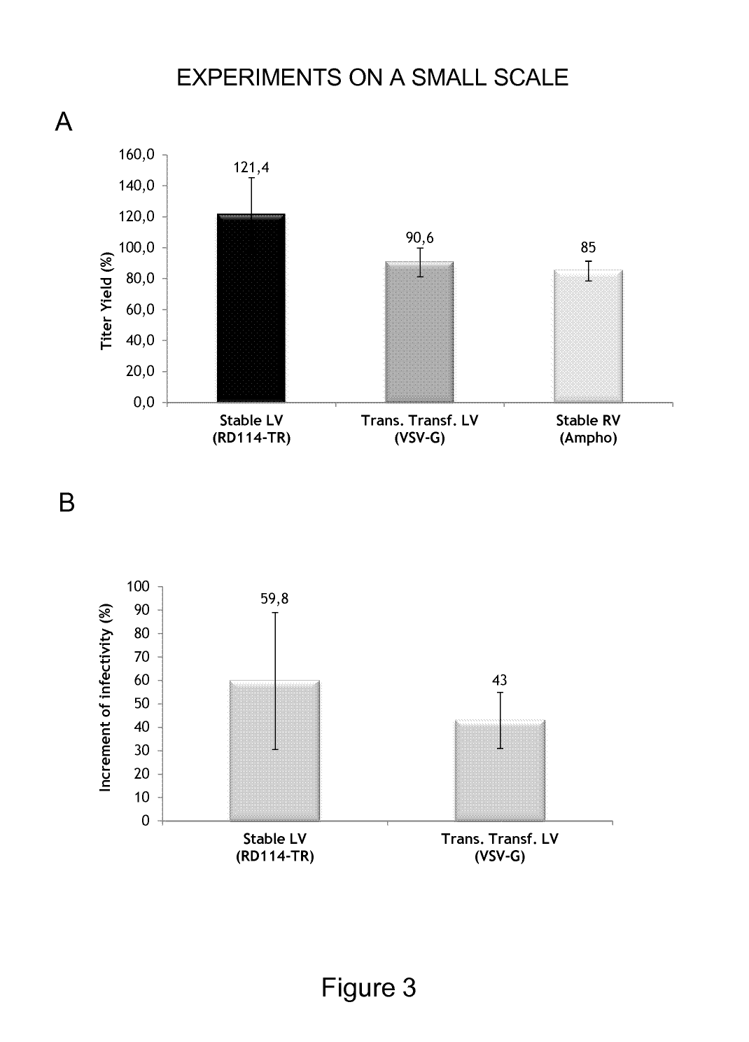

Each experiment was carried out at the following conditions: 1) diluted supernatant volume (one ml of supernatant diluted 1:5 with PBS/2% FCS/0.5% BSA); 2) supernatant:microbead suspension (vol:vol) ratio 1:40; 3) anti-LNGFR Abs directly coupled to magnetic beads (CD271 microbeads). The output of the analysis corresponds to both the percentage of titer yield (FIG. 3A) and the percentage of the increment of infectivity (FIG. 3B) of purified VV relative to that of unpurified magnetically labelled W. Titer calculation was performed on SupT1 cells, as detailed in Example III. Remarkably, the yield for LV produced stably was superior to 100% (121% on average) and that of LV produced transiently was 90%. This means that the purification of LV, regardless the type of envelope they mount, is highly effective in removing serum proteins or other contaminants that might decrease titer values. The purification yield of γRV is slightly inferior to that of LV (85%). Moreover, a considerable increase of infectivity of lentiviral vectors purified with the method of the invention has been obtained (43% and 60% for transient and stable production, respectively).

### Example V

### Purification of VV by the Anti-LNGFR Abs on a Large Scale

Large scale purification of VV was carried as follows. Filtered supernatants (0.45 μm) containing LV (800 ml) were concentrated 8-fold by centrifugation at low speed (3,400×g) for 16 hours at +4° C. in refrigerated bench top centrifuge. VV pellets were resuspended in 100 ml buffer PBS/EDTA 0.5% human serum albumin (HSA) and then incubated with anti-LNGFR Ab conjugated microbeads suspension (CD271 Microbeads, Miltenyi Biotec, cat. #130-091-330) at the 1:40 ratio (vol/vol) in a 150-ml transfer bag (Miltenyi Biotec cat. #183-01). The samples were then incubated at RT for 30 minutes on an orbital shaker. The magnetically labelled samples were loaded on the CliniMacs® Plus Instrument and the automated separation programme Enrichment 3.2 was started. The purified LV was recovered in 40 ml and an aliquot was evaluated for purification performance by potency calculation.

### Example VI

### Analysis of Potency of Purified Versus Unpurified VV in Large Scale Preparation

Two experiments were performed, using the 2^{nd} generation LV expressing the Chim3 transgene produced from the stable packaging cell line RD2-MolPack-Chim3.25. Results are summarized on Table 3 and 4. Each experiment was carried out as described in Example V. The output of the analysis corresponds to both the percentage of titer yield and the percentage of the increment of infectivity of purified LV relative to that of unpurified viral particles bound to anti-LNGFR Ab conjugated to the magnetic beads (Table 3) or relative to that of supernatant (Table 4). Titer calculation was performed on SupT1 cells as disclosed in example III. The titer yield of large scale purification (Table 3, EL/Input) was around 60% in the single step of separation of the complex viral vector-ligand. Before starting the separation phase, the viral supernatant is enriched in its viral titer through preliminary filtration and centrifugation steps, that roughly eliminate contaminants and transduction inhibitors, and also through the incubation with the ligand of the receptor. The final titer yield after the complete multi-step purification process (harvest viral supernatant vs final purified product) (Table 4, EL/Sup), is more than 100%: 118% for experiment 1 and 231% for experiment 2. Most importantly, the infectivity of purified particles is dramatically increased of three times in respect to the unpurified, with an even higher enrichment as compared to the small scale experiments indicating that large scale and automation further increase the yield of the process in terms of functionality of VV.

TABLE 1
Summary of experiments in small scale

| Type of Production | Vector | ΔLNGFR copy | Env | TU Yield (%)^{a} | Infect. (%)^{b} | Exp |
|---|---|---|---|---|---|---|
| Stable LV | LV | 20 | RD114-TR | 121.4 ± 24SEM | 59.8 ± 29SEM | 5 |
| Trans. Transf. LV^{c} | LV | nd | VSV-G | 90.6 ± 9.3SEM | 43.0 ± 12SEM | 5 |
| Stable RV | RV | 2 | e4070 | 85.0 ± 6.4SEM | nd | 6 |

Abbreviations: LV, lentiviral vector; RV, retroviral vector; RD114-TR, chimeric envelope from the feline endogenous retrovirus and the TR domain of MLV env; VSV-G, vesicular stomatitis virus envelope glycoprotein G
^{a}Yield has been calculated as the % of total TU recovered respect to the total TU input loaded into the magnetic column.
^{b}Infectivity has been calculated as the % of increment of infectivity of purified versus inputVV.
^{c}Transient transfection has been carried out on HEK293T cells as described in the text.

TABLE 2
Summary of all experiments in small scale

| Number of exp. | | | | | | | | | | | | | | |
|---|---|---|---|---|---|---|---|---|---|---|---|---|---|---|
| EX1 | | | EX2 | | | EX3 | | | EX4 | | | EX5 | | |
| Ti- | | In- | Ti- | | In- | Ti- | | In- | Ti- | | In- | Ti- | | In- |
| ter^{a} | p24Gag^{b} | fect.^{c} | ter^{a} | p24Gag^{b} | fect.^{c} | ter^{a} | p24Gag^{b} | fect.^{c} | ter^{a} | p24Gag^{b} | fect.^{c} | ter^{a} | p24Gag^{b} | fect.^{c} |

Stable LV (RD114-TR)

| | | | | | | | | | | | | | | | |
|---|---|---|---|---|---|---|---|---|---|---|---|---|---|---|---|
| Input | 4.0 × | 1.3 | 3.0 × | 4.5 × | 1.2 | 3.8 × | 5.2 × | 1.5 | 3.4 × | 5.2 × | 1.5 | 3.4 × | 7.9 × | 1.5 | 5.2 × |
| | 10³ | | 10³ | 10³ | | 10³ | 10³ | | 10³ | 10³ | | 10³ | 10³ | | 10³ |
| Flow | 0 | 0.1 | nd | 1.2 × | nd | nd | 0 | 0.3 | nd | 1.9 × | 0.28 | 6.8 × | 3.0 × | 0.3 | 1.0 × |
| through | | | | 10³ | | | | | | 10³ | | 10³ | 10² | | 10³ |
| Eluted | 8.2 × | 1.1 | 7.5 × | 3.7 × | 0.8 | 4.6 × | 6.3 × | 1.2 | 5.2 × | 7.3 × | 1.1 | 6.6 × | 5.1 × | 1.2 | 4.2 × |
| | 10³ | | 10³ | 10³ | | 10³ | 10³ | | 10³ | 10³ | | 10³ | 10³ | | 10³ |
| YIELD: | nd^{d} | 7.6^{d} | nd^{e} | 26 | nd | nd | nd | 20 | nd | 36 | 18 | 50 | 3.7 | 20 | −80 |
| % rec^{d}. & | | | | | | | | | | | | | | | |
| % var. FT/ | | | | | | | | | | | | | | | |
| In^{e} | | | | | | | | | | | | | | | |
| YIELD: | 200^{f} | 84^{f} | 150^{g} | 82 | 66 | 21 | 121 | 80 | 53 | 140 | 73 | 94 | 64 | 80 | −19 |
| % rec.^{f} & | | | | | | | | | | | | | | | |
| % var. EL/ | | | | | | | | | | | | | | | |
| In^{g} | | | | | | | | | | | | | | | |

Transient Transfection LV (VSV-G)

| | | | | | | | | | | | | | | | |
|---|---|---|---|---|---|---|---|---|---|---|---|---|---|---|---|
| Input | 1.4 × | 165 | 8.4 × | 1.3 × | 160 | 8.1 × | 1.4 × | 187 | 7.4 × | 1.7 × | 196 | 8.6 × | 1.7 × | 196 | 8.6 × |
| | 10⁷ | | 10⁴ | 10⁷ | | 10⁴ | 10⁷ | | 10⁴ | 10⁷ | | 10⁴ | 10⁷ | | 10⁴ |
| Flow | 2.1 × | 44 | 4.9 × | 2.0 × | 22 | 9.0 × | 5.0 × | 81 | 6.1 × | 7.2 × | 91 | 7.9 × | 5.6 × | 81 | 6.9 × |
| through | 10⁶ | | 10⁴ | 10⁶ | | 10⁴ | 10⁶ | | 10⁴ | 10⁶ | | 10⁴ | 10⁶ | | 10⁴ |
| Eluted | 1.7 × | 101 | 1.6 × | 1.0 × | 100 | 1.0 × | 1.1 × | 110 | 1.0 × | 1.3 × | 105 | 1.2 × | 1.2 × | 108 | 1.1 × |
| | 10⁷ | | 10⁵ | 10⁷ | | 10⁵ | 10⁷ | | 10⁵ | 10⁷ | | 10⁵ | 10⁷ | | 10⁵ |
| YIELD: | 15 | 26 | −41 | 15 | 14 | 10 | 36 | 43 | −17 | 42 | 46 | −8 | 33 | 41 | −20 |
| % rec^{d}. & | | | | | | | | | | | | | | | |
| % var. FT/ | | | | | | | | | | | | | | | |
| In^{e} | | | | | | | | | | | | | | | |
| YIELD: | 121 | 61 | 90 | 76 | 62 | 23 | 78 | 59 | 35 | 76 | 53 | 39 | 70 | 55 | 28 |
| % rec.^{f} & | | | | | | | | | | | | | | | |
| % var. EL/ | | | | | | | | | | | | | | | |
| In^{g} | | | | | | | | | | | | | | | |

| Stable RV (Ampho) | | | | | | | | |
|---|---|---|---|---|---|---|---|---|
| Number of exp. | | | | | | | | |
| EX1 | | | EX2 | | | EX3 | | |
| Titer | p19Gag | Infect. | Titer | p19Gag | Infect. | Titer | p19Gag | Infect. |

| | | | | | | |
|---|---|---|---|---|---|---|
| Input | 6.0 × | nd | 8.0 × | nd | 9.3 × | nd |
| | 10⁴ | | 10⁴ | | 10⁴ | |
| Flow | 7.6 × | | 5.4 × | | 1.5 × | |
| through | 10³ | | 10³ | | 10⁴ | |
| Eluted | 6.9 × | | 6.8 × | | 5.8 × | |
| | 10⁴ | | 10⁴ | | 10⁴ | |

| | | | |
|---|---|---|---|
| YIELD: | 12 | 6.7 | 16 |
| % rec^{d}. & | | | |
| % var. FT/ | | | |
| In^{e} | | | |
| YIELD: | 115 | 85 | 62 |
| % rec.^{f} & | | | |
| % var. EL/ | | | |
| In^{g} | | | |

| Stable RV (Ampho) | | | | | | | | |
|---|---|---|---|---|---|---|---|---|
| Number of exp. | | | | | | | | |
| EX4 | | | EX5 | | | EX6 | | |
| Titer | p19Gag | Infect. | Titer | p19Gag | Infect. | Titer | p19Gag | Infect. |

| | | | | | | |
|---|---|---|---|---|---|---|
| Input | 9.3 × | nd | 7.5 × | nd | 7.5 × | nd |
| | 10⁴ | | 10⁴ | | 10⁴ | |
| Flow | 3.0 × | | 1.5 × | | 1.4 × | |
| through | 10⁴ | | 10⁴ | | 10⁴ | |
| Eluted | 8.1 × | | 6.1 × | | 6.0 × | |
| | 10⁴ | | 10⁴ | | 10⁴ | |

| | | | |
|---|---|---|---|
| YIELD: | 32 | 20 | 19 |
| % rec^{d}. & | | | |
| % var. FT/ | | | |
| In^{e} | | | |
| YIELD: | 87 | 81 | 80 |
| % rec.^{f} & | | | |
| % var. EL/ | | | |
| In^{g} | | | |

^{a}Titer of the total amount of loaded and eluted VV
^{b}Total amount of p24Gag expressed in ng
^{c}Infectivity expressed as TU/ng p24Gag
^{d}Percentage of total titer and p24Gag in the flow thorugh material respect to the total titer and p24Gag of the input VV
^{e}Percentage of variation of the infectivity in the flow through material respect to the infectivity of the input VV
^{f}Percentage of total titer and p24Gag in the eluted material respect to the total titer and p24Gag of the input VV
^{g}Percentage of variation of the infectivity in the eluted material respect to the infectivity of the input VV
nd: not determined

TABLE 3
Summary of large scale experiments

| | Stable LV (RD114-TR) | | | | | | | |
|---|---|---|---|---|---|---|---|---|
| | Number of exp. | | | | | | | |
| | EX1 | | | | EX2 | | | |
| | Titer^{a} | p24Gag^{b} | Infect.^{c} | Inf/Tot^{d} | Titer^{a} | p24Gag^{b} | Infect.^{c} | Inf/Tot^{d} |
| Input (102.5 ml) | 6.1 × 10⁷ | 6,700 | 7.0 × 10³ | 1:1,098 | 8.6 × 10⁷ | 6,060 | 1.4 × 10⁴ | 1:704 |
| Eluted (40 ml) | 3.9 × 10⁷ | 1,480 | 2.6 × 10⁴ | 1:358 | 5.2 × 10⁷ | 1,300 | 4.0 × 10⁴ | 1:250 |
| YIELD: % rec^{e} & % var.^{f} of EL/IN | 64^{e} | 22^{e} | 371^{f} | | 60^{e} | 21^{e} | 285^{f} | |

^{a}Titer of the total amount of loaded and eluted LV
^{b}Total amount of p24Gag expressed in ng
^{c}Infectivity expressed as TU/ng p24Gag
^{d}Ratio between total infectious vs physical particles
^{e}Percentage of total titer and p24Gag in the eluted material respect to the total titer and p24Gag of the input LV, rec recovery
^{f}Percentage of variation of the infectivity in the eluted material respect to the infectivity of the input LV, var variation

TABLE 4
Large scale full process yield

| | Stable LV (RD114-TR) | | | | | | | |
|---|---|---|---|---|---|---|---|---|
| | Number of exp. | | | | | | | |
| | EX1 | | | | EX2 | | | |
| | Titer^{a} | p24Gag^{b} | Infect.^{c} | Inf/Tot^{d} | Titer^{a} | p24Gag^{b} | Infect.^{c} | Inf/Tot^{d} |
| Sup. (800 ml) | 3.3 × 10⁷ | 11,600 | 2.8 × 10³ | 1:3,300 | 2.2 × 10⁷ | 11,700 | 1.8 × 10³ | 1:5,318 |
| Eluted (40 ml) | 3.9 × 10⁷ | 1,480 | 2.6 × 10⁴ | 1:358 | 5.2 × 10⁷ | 1,300 | 4.0 × 10⁴ | 1:250 |
| YIELD: % rec^{e} & % var.^{f} of EL/SUP | 118^{e} | 13^{e} | 928^{f} | | 231^{e} | 11^{e} | 2222^{f} | |

^{a}Titer of the total amount of starting and eluted LV
^{b}Total amount of p24Gag expressed in ng
^{c}Infectivity expressed as TU/ng p24Gag
^{d}Ratio between total infectious vs physical particles
^{e}Percentage of total titer and p24Gag in the eluted material respect to the total titer and p24Gag of the input LV, rec recovery
^{f}Percentage of variation of the infectivity in the eluted material respect to the infectivity of the input LV, var variation

### REFERENCES

1. Baekeland, V., et al. (2003) Optimized lentiviral vector production and purification procedure prevents immune response after transduction of mouse brain. Gene Ther 10: 1933-1940.
2. Tuschong, L., et. al. (2002). Immune response to fetal calf serum by two adenosine deaminase-deficient patients after T cell gene therapy. Hum. Gene Ther. 13: 1605-1610.
3. Andreadis, S. T., et al. (1999). Large scale processing of recombinant retrovirus for gene therapy. Biotechnolo. Prog. 15: 1-11.
4. Lyddiatt, A., and O'Sullivan, D. A. (1998). Biochemical recovery and purification of gene therapy vectors. Curr. Opin Biotechnol. 9: 177-185.
5. Rodrigues, et al. (2007). Purification of retroviral vectors for clinical application: biological implications and technological challenges. J. of Biotech. 127: 520-541.
6. Arthur, L. O, et al. (1992). Cellular proteins bound to immunodeficiency viruses: implication for pathogenesis and vaccines. Science 258: 1935-1938.
7. Roberts, B. D., et al. (1999). Host protein incorporation is conserved among diverse HIV-1 subtypes. AIDS 13: 425-427.
8. Lawn, S. D., et al. (2000). Cellular compartments of human immunodeficiency virus type 1 replication in vivo: determination by presence of virion-associated host proteins and impact of opportunistic infection. J Virol 74 (1): 139-145
9. Verzeletti, S., et al. (1998). Herpes simplex virus thymidine kinase gene transfer for controlled graft-versus-host disease and graft-versus-leukemia: clinical follow-up and improved new vectors. Hum. Gene Ther, 9(15):2243-51
10. Sandrin, V., et al. (2002). Lentiviral vectors pseudotyped with a modified RD114 envelope glycoprotein show increased stability in sera and augmented transduction of primary lymphocytes and CD34+ cells derived from human and nonhuman primates. Blood 100: 823-832.
11. Porcellini, S., et al. (2009). The F12-Vif derivative Chim3 inhibits HIV-1 replication in CD4+T lymphocytes and CD34+-derived macrophages by blocking HIV-1 DNA integration. Blood 113: 3443-3452.
12. Porcellini, S., et al. (2010). Chim3 confers survival advantage to CD4+ T cells upon HIV-1 infection by preventing HIV-1 DNA integration and HIV-1-induced G2 cell-cycle delay. Blood 115: 4021-4029.
13. Bastiani Lallos, L., Laal, S., Hoxie, J. A., Zolla-Pazner, S., and Bandres, J. C. (1999) Exclusion of HIV coreceptor CXCR4, CCR5, and CCR3 from the HIV envelope. AIDS Research and Human Retroviruses 15: 895-897.
14. Martens et al., (2011). Large-Scale manufacture and characterization of a lentiviral vector produced for clinical ex vivo gene therapy application. Hum. Gene Ther 22:343-356.
15 Patrick Salmon and Didier Trono, (2006) Current Protocols in Neuroscience Supplement 37: 4.21.1-4.21.24, John Wiley & Sons, Inc

1. A method for the purification of a viral vector comprising:
i. introducing an exogenous gene encoding a cell surface marker and a gene of interest in a packaging cell line;
ii. culturing the so obtained producer cell line;
iii. collecting the supernatant containing viral vector particles bearing the cell surface marker on their external envelope;
iv. v incubating said supernatant with a ligand able to bind to the cell surface marker,
v. separating complex ligand-viral vector; and
vi. obtaining purified viral vector particles.

2. The method according to claim 1, wherein the viral vector is selected from the group consisting of: a retroviral vector, a lentiviral vector, an alpha viral vector, a rhabdoviral vector, and a orthomyxoviral vector.

3. The method according to claim 1, wherein the cell surface marker is selected from the group consisting of: CD26, CD36, CD44, CD3, CD25, and the truncated form of Low Nerve Growth Factor Receptor (ΔLNGFR).

4. The method according to claim 1, wherein the cell surface marker is the truncated form of Low Nerve Growth Factor Receptor (ΔLNGFR).

5. The method according to claim 1, wherein the expression of the cell surface marker is transient.

6. The method according to claim 1, wherein the expression of the cell surface marker is stable.

7. The method according to claim 1, wherein the gene of interest and the exogenous gene are expressed in the same transfer vector.

8. The method according to claim 1, wherein the gene of interest and the exogenous gene are expressed in separate vectors.

9. The method according to claim 1, wherein the ligand is a chemical or a biological entity selected from the group consisting of: an agonist, an antagonist, a peptide, a peptidomimetic, an antibody, an antibody fragment, and an affibody.

10. The method according to claim 1, wherein the ligand is linked to a moiety that can be separated from the supernatant.

11. The method according to claim 9, wherein the ligand is an antibody conjugated to magnetic beads, and wherein separation is obtained by applying a magnetic field to a solution containing the complex antibody-viral vector.

12. The method according to claim 11, wherein purified viral vector is obtained by removing the magnetic field.

13. The method according to claim 1, wherein the separation of the complex antibody-viral vector is performed on a column.

14. An exogenous cell surface marker expressed in a packaging cell line for use in the purification of viral vectors produced by said packaging cell line.

15. The exogenous cell surface marker according to claim 14, wherein said marker is selected from the group consisting of: CD26, CD36, CD44, CD3, CD25, and the truncated form of Low Nerve Growth Factor Receptor (ΔLNGFR).

16. The exogenous cell surface marker according to claim 14, wherein said marker is ΔLNGFR.
